(19) United States
(12) Patent Application Publication
(10) Pub. No.: US 2023/0016459 A1
(43) Pub. Date: Jan. 19, 2023

(54) OPTICAL COMPONENTS FOR ENDOSCOPE COMPANION DEVICES

(71) Applicant: GI Scientific, LLC, Arlington, VA (US)

(72) Inventor: Scott Miller, Arlington, VA (US)

(21) Appl. No.: 17/786,296

(22) PCT Filed: Dec. 16, 2020
(86) PCT No.: PCT/US20/65424

§ 371 Date: Jun. 16, 2022

Related U.S. Application Data
(60) Provisional application No. 62/949,238, filed on Dec. 17, 2019.

(51) Int. Cl. A61B 1/00; A61B 1/018; A61B 1/06; A61B 1/05

(52) U.S. Cl. CPC A61B 1/00098; A61B 1/00142; A61B 1/018; A61B 1/0676; A61B 1/05

(57) ABSTRACT

A coupler device for an endoscope that includes a main body having a proximal end configured to attach to the distal end portion of the endoscope, and a protective cover to reduce the ingress of debris, fluid, bacteria, or other unwanted matter from the working end of the endoscope which could lead to infection and decreased performance of the scope. The coupler device further includes a visualization section on the main body between the camera lens and light source of the endoscope and the target site within the patient. The visualization section includes one or more optical components configured to reduce an amount of reflected light from the surface and/or to inhibit condensation of water droplets on the surface. Reducing the glare and/or fogging of the visualization section significantly improves the surgeon's view of the target site through the optical coupler.

### CROSS-REFERENCE TO RELATED APPLICATIONS

This application is the National Stage of International Application No. PCT/US2020/065424 filed Dec. 16, 2020 which claims the benefit of U.S. Provisional Application No. 62/949,238, filed Dec. 17, 2019, the entire disclosure of which is incorporated herein by reference for all purposes.

### FIELD

The present disclosure generally relates to companion devices, such as optical couplers, for use with endoscopes, and more particularly to optical couplers that have optical components for reducing reflected light and/or inhibiting water condensation on the camera lens of the endoscope.

### BACKGROUND

Recent advances in optical imaging technology have allowed many medical procedures to be performed today in a minimally invasive manner. The evolution of the more sophisticated, flexible scope with advanced visual capabilities has allowed access to regions deep within the human body that could only be achieved before with invasive surgical intervention. This modern day convenience has resulted in an increase in the demand for, as well as the number of, endoscopic, laparoscopic, arthroscopic, ophthalmoscopic, or other remote imaging visualization procedures performed every year in the U.S and globally. While these procedures are relatively safe, they are not without risks.

Endoscopy, for instance, is a procedure in which a lighted visualization device called an endoscope is inserted into the patient's body to look inside a body cavity, lumen or organ, or combination, for the purpose of examination, diagnosis or treatment. The endoscope may be inserted through a small incision or through a natural opening of the patient. In a bronchoscopy, the endoscope is inserted through the mouth, while in a sigmoidoscopy, the endoscope is inserted through the rectum. Unlike most other medical imaging devices, endoscopes are inserted directly into the organ, body cavity or lumen.

Today, most endoscopes are reused. This means that, after an endoscopy, the endoscope goes through a cleaning, disinfecting or sterilizing, and reprocessing procedure to be introduced back into the field for use in another endoscopy on another patient. In some cases, the endoscope is reused several times a day on several different patients.

While the cleaning, disinfecting and reprocessing procedure is a rigorous one, there is no guarantee that the endoscopes will be absolutely free and clear of any form of contamination. Modern day endoscopes have sophisticated and complex optical visualization components inside very small and flexible tubular bodies, features that enable these scopes to be as effective as they are in diagnosing or treating patients. However, the tradeoff for these amenities is that they are difficult to clean because of their small size, and numerous components. These scopes are introduced deep into areas of the body which expose the surfaces of these scopes to elements that could become trapped within the scope or adhere to the surface, such as body fluids, blood, and even tissue, increasing the risk of infection with each repeated use.

Endoscopes used in the gastrointestinal tract, such as endoscopic ultrasound scopes (EUS) and duodenoscopes with a side-viewing capability, have an added complexity in that they are in a bacteria rich environment. Typical duodenoscopes and EUS scopes have internal moving components like an elevator with hinges attached to a cable for actuation. The elevator is used to deflect and therefore change the direction of instruments passed down the scope's working channel. This elevator is beneficial in that it can allow the user to change the direction of a wire or a catheter to direct the wire or catheter into a specific opening, so that one or more instruments can be turned to enter a particular body lumen or to penetrate or sample tissue. However, given the size, location and movement of the elevator during use, the elevator creates cleaning issues, including the risk that bacteria finds its way into the elevator's hinges and other hard to clean locations on the scope. This provides an opportunity for bacteria to colonize and become drug resistant, creating the risk of significant illness and even death for a patient. This infection risk is also present in the cable mechanisms that are used to move the elevator mechanism back and forth and in other aspects of current scope designs. Moreover, in addition to the health risks posed by bacterial contamination, the accumulation of fluid, debris, bacteria, particulates, and other unwanted matter in these hard to clean areas of the scope also impact performance, shortening the useful life of these reusable scopes.

To reduce infection risks and protect the working end of endoscopes, disposable optical coupler devices have been designed for covering and at least partially sealing a portion of existing endoscopes. These coupler devices typically attach to the working end of the endoscope and have a visualization section composed of an optical material, such as glass, polycarbonate, acrylic, a clear gel or silicone, or other material with sufficient optical clarity to transmit an image, and which generally align with the camera lens and light source of the scope to allow for light to pass through the visualization section to provide a view of the target site by the endoscope.

One of the drawbacks with existing coupler devices is that fluid in or around the target site tends to build up and condense into droplets on the surfaces of the visualization section, causing these surfaces to “fog” and limit the view of the lumen, organ, specific tissue, surgical site or other desired visualization point. This may occur between the scope and the optical coupler device or an the external surface of the optical coupler device, or in combination.

Another drawback with existing coupler devices is that light passing through the visualization section of the device tends to reflect from tissue surfaces in the patient back to the camera lens or the light source. This phenomenon may occur, for example, when the operator advances the endoscope from an area of smaller volume in the patient, such as the intestines, to an area of larger volume, such as an organ (e.g., the stomach). The larger volume area will cause a larger amount of reflected light passing back into the coupler device and onto the camera lens of the scope. This reflected light creates a glare that further limits the ability of the operator to view the target site.

Accordingly, it is desirable to provide devices which serve as convenient accessories for currently existing endoscopes to reduce the risk of contamination and infection, while also improving the performance of the endoscope. It is particularly desirable to provide an endoscope accessory or companion device, such as an optical coupler, for the working end of an endoscope that provides a clear view of the desired observation point in the patient's anatomy with minimal glare and/or fogging on the light source or the camera lens of the endoscope.

### SUMMARY

The present disclosure provides a coupler device for covering and at least partially sealing a portion of the working end of an endoscope. The coupler device protects the scope and its components, particularly the scope elevator, to reduce the risk of debris, fluid and other matter ending up in the elevator and behind the elevator and the working channel and other hard-to-clean areas, potentially causing infection risk. The coupler device further includes one or more optical components configured to reduce an amount of reflected or stray light from the surfaces of the optical coupler and/or surrounding tissue surfaces. The coupler device may also include one or more optical components that inhibit condensation of water droplets on the optical coupler, the camera lens, or both. Reducing the glare and/or fogging of the optical coupler significantly improves the surgeon's view of the target site through the endoscope.

In one embodiment, a coupler device according to the present disclosure includes a main body comprising an at least partially closed distal end and a proximal end configured for attachment to a distal end portion of the endoscope. The coupler device further includes a substantially transparent visualization section between the light and the camera lens of the endoscope and the target site to allow viewing of the target site. The visualization section or the main body of the coupler device includes an optical component on at least one of its surfaces, or within the visualization section. The optical component is configured to reduce an amount of reflected light from the surface(s) of the visualization section or the surrounding tissue surfaces to reduce the glare and improve the view of the observation site.

The visualization section may comprise a substantially transparent film, sheet, layer, coating, wall or other substrate positioned on the coupler device so as to allow light to pass therethrough. Alternatively, the visualization section may comprise a transparent area within the main body of the optical coupler comprising a material capable of transmitting an optical image, such as a clear gel or other substantially transparent material that will allow light to pass therethrough. In certain embodiments, the camera lens and light guide are directed laterally from the shaft of the endoscope (i.e., side viewing endoscopes) and the visualization section is disposed on a lateral surface of the coupler device such that light from the light guide passes therethrough to the target site. In other embodiments, the camera lens and light guide may be directed longitudinally from the shaft of the endoscope (i.e., end viewing scopes) and the visualization section is disposed on a distal surface of the coupler device. In still other embodiments, the coupler device or the endoscope may comprise mirrors or other light reflecting surfaces to redirect light passing to and from the camera and light guide so as to “align” the visualization section with the light guide and camera lens. In this latter embodiment, the visualization section may not necessarily be directly opposite the camera and light guide so long as the mirrors or other redirecting surfaces are configured to pass the light through the visualization section to the target site.

The visualization section can be any optical material capable of transmitting an optical image, such as polycarbonate, glass, clear gel or the like. In one embodiment, the visualization section comprises polycarbonate and the optical component comprises a layer of different material than polycarbonate, preferably having a different refractive index than polycarbonate. The optical layer creates an interference effect with the polycarbonate, thereby reducing the total percentage of reflected light without substantially reducing the transparency of the visualization section.

The optical layer may be disposed on the inner surface, the outer surface or on both the inner and outer surfaces of the visualization section. Alternatively, the optical layer may be disposed within the visualization section. In other embodiments, the optical layer may be disposed on the surface of the camera lens and/or light source. In one embodiment, the optical layer comprises an anti-reflective coating applied to the inner surface of the visualization section that reduces a substantial portion of the reflected light in a visible range of light waves (i.e., between about 400 to 700 nanometers). In a preferred embodiment, the anti-reflective coating comprises magnesium fluoride.

In other embodiments, the optical layer comprises two or more anti-reflecting coatings. Preferably, at least one of the coatings comprises magnesium fluoride. Other suitable coatings may include zinc oxide, aluminum oxide, cerium trifluoride, or the like. In an exemplary embodiment, the optical layer comprises sequential layers of coatings, such as cerium fluoride, zinc oxide and magnesium fluoride.

In yet another embodiment, the optical component may comprise a light absorbing material positioned in, or on, one or more areas of the coupler device, such as the visualization section, the main body, one of the outer surfaces of the main body, the open area over the camera lens and light guide, within the flexible working channel region, such as within, or on, the working channel extension or the flexible membrane. The light absorbing material is positioned to absorb reflected, scattered or stray light from the patient's tissue and/or the surfaces of the optical coupler, thereby minimizing glare that may otherwise be caused by this reflected light. The light absorbing material may be a thin black strip, square, circle, rectangle or other suitable shape positioned to absorb reflected light such that the light does not interfere with the camera lens.

Alternatively, the optical component may comprise a baffle positioned on, or within, the coupler device. The baffle preferably includes one or more channels or vanes positioned and configured to direct stray reflected light away from the camera lens on the endoscope. Baffles can be designed to shield the light coming from sources outside of the field of view (FOV) of the camera lens. The light outside the angular view of the system must execute multiple number of reflections on its surfaces, minimizing the intensity of the light that effectively reach the camera lens. The internal surfaces may comprise light absorbing materials or colors, e.g., blackened surfaces.

In another aspect of the invention, a coupler device for an endoscope having a light and a camera lens comprises a main body comprising an at least partially closed distal end and a proximal end configured for attachment to a distal end portion of the endoscope. The coupler device further includes a substantially transparent visualization section between the light and the camera lens of the endoscope and the target site to allow viewing of the target site with the scope. The visualization section includes an optical layer on at least one of its surfaces, or within the visualization section. The optical layer is configured to inhibit condensation of fluid, such as water, on the surface(s) of the visualization section. The optical layer reduces “fogging’ on, or within, the visualization section, thereby improving the view of the target site.

In certain embodiments, the optical layer comprises one or more chemicals applied to, or within, the visualization section. The chemicals are configured to inhibit condensation of water droplets on an outer surface of the visualization section (i.e., the surface facing the target site). In other embodiments, the optical layer comprises an anti-fogging film applied to the outer surface of the visualization section and in other embodiments the application is to the inside surface of the visualization section, or on both the outside and inside surfaces.

In another embodiment, the optical layer comprises a surfactant configured to minimize the surface tension of water on the outer surface of the visualization section. The surfactant may comprise nonionic, anionic, cationic or amphoteric polarities and may include detergents, fatty acid soaps, dispersants or other suitable materials that reduces the force of surface tension from water molecules on the visualization section to thereby reduce the number of water droplets that form on the outer surface.

In another embodiment, the optical layer comprises a hydrophilic coating that maximizes the surface energy on an outer surface of the visualization section. Suitable hydrophilic coatings may include demisters, defoggers, defrosters, polymers and hydrogels, such as gelatin, colloids and nanoparticles, such as titanium dioxide, or other suitable hydrophilic materials.

In another aspect of the invention, a coupler device for an endoscope having a light and a camera lens comprises a main body comprising an at least partially closed distal end and a proximal end configured for attachment to a distal end portion of the endoscope. The coupler device further includes a visualization section between the light and the camera lens of the endoscope and the target site to allow viewing of the target site with the scope. The coupler device further includes a first optical component on, or within, the visualization section configured to reduce reflected light and a second optical component on, or within, the visualization section configured to inhibit condensation of fluid on the visualization section.

In certain embodiments, the coupler device includes an open area, cavity or channel coupled to a working channel of the endoscope that allows an instrument to pass through the coupler device to the desired observation site seen through the coupler and the scope. The instrument(s) may be articulated by a variety of suitable means, such as cables, elevators, piezo electric materials, micro motors, organic semiconductors, electrically activated polymers or other sources of energy or power, that are either disposed within the coupler device, on or within the endoscope, or external to both and suitably coupled to the instrument(s).

In other embodiments, the coupler device includes a flexible working channel extension that extends the working channel of the scope and can be angularly adjustable. The flexible working channel extension may be adjustable by an elevator or cable passing through the endoscope. Alternatively, the coupler device may include its own actuator, such as an elevator, cable, or similar actuation means, for adjusting the working channel extension and thereby articulating instruments passing through the endoscope. The actuator may be powered by any suitable source of energy, such as a motor or the like. The source of energy may be coupled to the actuator either directly through the scope, or indirectly through magnetic, electric, or some other source of energy. The source of energy may be disposed within the coupler device, or it may be external to the coupler device (i.e., either disposed on the proximal end of the scope or external to the patient).

In another aspect of the invention, a system includes an optical coupler as described above with an endoscope, such as a side-viewing scope such as a duodenum scope, endoscopic ultrasound scope (EUS) or the like. The side-viewing scope includes a working channel, a light source and a camera. One or more optical layers are disposed on the surfaces of either the light source, the camera or both. The optical layers may include any of the anti-fogging layers and/or the anti-reflective layers described above. In this embodiment, the optical layers are disposed on the endoscope itself, rather than the coupler device, in order to reduce glare and/or condensation within the coupler device (i.e., between the camera lens and light source and the visualization section of the coupler device).

The scope may further comprise an actuator for adjusting the angle of the working channel extension of the optical coupler. In one embodiment, the actuator comprises an elevator disposed within a distal end portion of the scope. In another embodiment, the actuator comprises a cable extending through the scope. In these embodiments, the coupler device is configured to cooperate with the scope's actuator or cable to articulate instruments through the coupler device. In other embodiments, the coupler device includes its own actuator for articulating instruments, eliminating the need to have a scope with an elevator or cable actuator. In yet other embodiments, the working channel extension of the coupler device does not articulate.

The coupler device may be provided as a single-use disposable accessory to an endoscope that provides the user with the ability to change the angle of exit of a device being advanced out of the working channel of an endoscope, without exposing the distal end of the scope to bacteria, debris, fluid and particulate matter. In some embodiments, the device attaches to the end of the endoscope and covers the working channel of the endoscope with a working channel extension in the coupler device, allowing an instrument to be passed down the working channel of the endoscope and into the working channel extension of the coupler device. The working channel extension can provide a seal against the scope working channel, so instruments can be passed back and forth through the scope working channel and out the working channel extension of the coupler device without fluid and bacteria entering areas outside of the scope working channel. This seal is accomplished, in some embodiments, through an extension of the device working channel into the scope working channel, through a gasket on the end of the working channel extension, by way of a temporary glue, through pressure and the seal of the overall device against the distal end of the scope, through the selection of elastic and elastomeric materials, and other suitable and alternative means.

In some embodiments, the device allows the user to articulate the working channel of the device in the direction preferred by the user of the endoscope, so that a wire, catheter or other instrument being advanced down the working channel of the endoscope can direct the wire or catheter or other instrument in a preferred direction different than the angle at which the instrument would exit the endoscope if the coupler device was not in place or if an elevator in the scope is not used. This redirection of an instrument has the benefit of assisting with the navigation of the device, while not allowing fluid, debris, particulate matter, bacteria and other unwanted elements to enter hard to clean areas of the endoscope, especially at the distal end of the endoscope.

The benefits of the invention include allowing the physician to change the angle of exit, so that one or more devices can be turned to enter a particular body lumen, such as a biliary duct or pancreatic duct, or other hard to reach area, including in non-medical procedures, while sealing the distal end of the scope to prevent infection and the intrusion of debris and particulate matter into interior elements of the scope that are hard to reach to effectively clean.

In some embodiments, the device may be formed of an optically clear material that covers the end of the endoscope and seals the end of the endoscope, allowing visualization of the endoscope's camera without obscuring the view by the device. The optically clear material may also cover the endoscope's light guide to allow the light projected by the endoscope to illuminate the field of view of the endoscope. In some embodiments, the optically clear material may include navigation markers to orient the user when visualizing tissue, such as markers to identify the relative position of the scope as the user visualizes the tissue through the optically clear material.

In some embodiments, the device may have multiple cables so the angle of exit can be articulated in multiple directions, including in different quadrants, unlike with the current endoscope elevators, which can only deflect and therefore redirect an instrument in a single axis due to the limited travel of endoscope elevators, which can only be raised or lowered, but not moved from side to side or articulated into other quadrants. In some embodiments, the cable(s) may be attached directly to the working channel extension or to other devices that can be articulated and cause the working channel extension to change its angle of exit, including, for example, a dowel underneath the working channel extension, but encased in the device that can be advanced forward and backward to move the working channel extension as the cable is advanced and retracted. In some embodiments, the articulation ability of the coupler device may be created with an elevator embedded in the coupler device, which is disposable and therefore thrown away after the procedure. Alternatively, the working channel extension may be fixed such that it does not articulate.

The articulation ability of the coupler device may also take place with elements that do not involve cables, including for example, piezo electric materials, micro motors, organic semiconductors, and electrically activated polymers. In some embodiments, the articulation ability of the coupler device may also take place with the transfer of force to the working channel extension or an embedded elevator through interlocking connectors that transfer force, wires that twist, slidable sheaths, and memory metals that change shape through the transfer of temperature. In some embodiments, the device includes a power connector or motors to deliver energy, including electromagnetic energy, to the device to cause a transfer in force to change the angle of exit from the coupler device as an instrument is passed through the device, or in advance of passing an instrument through the device. This transfer of force can include causing the device to rotate as it exits the working channel extension. The device may be navigated and articulated by the user directly, or as part of a robotic system in which the users input is translated through the system through various means, including cables, power connectors, motors, electromagnetic energy, slidable sheaths, haptics, computer-guided and directed input, and other means to direct and guide the device to its intended location, including to specific diagnosis and treatment objectives in a patient, or in non-medical applications, to a desired remote location.

In some embodiments, the device may be integrated into a scope and configured to be detachable and reusable for separate cleaning, including manual cleaning, in an autoclave, an ETO sterilizer, gamma sterilizer, and other sterilization methods.

In some embodiments, the coupler device may cover the entire distal end of the endoscope, or may just cover hard to clean areas. In some embodiments, the coupler device may cover the distal end of the endoscope, or a portion thereof, or it may include a sheath attached to the coupler device which covers the entirety of the scope that is exposed to fluid, debris, particulate matter, bacteria and other unwanted elements.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a partial cross-sectional view of the proximal portion of a representative endoscope according to the present disclosure;

FIG. 2 is a perspective view of the distal end portion of a side-viewing endoscope according to the present disclosure;

FIGS. 3A and 3B are isometric views of an exemplary embodiment of the coupler device of the present disclosure in use with a duodenum scope.

FIGS. 4A and 4B illustrate partial cutaway views of the coupler device and a duodenum scope of FIGS. 3A and 3B, respectively.

FIG. 5 illustrates another cutaway view of the coupler device and a duodenum scope of FIGS. 3A and 3B.

FIG. 6 illustrate yet another cutaway view of the coupler device and a duodenum scope of FIGS. 3A and 3B.

FIG. 7 is a cutaway side view of the coupler device and a duodenum scope of FIGS. 3A and 3B in a first position.

FIG. 8 is a cutaway side view of the coupler device and a duodenum scope of FIGS. 3A and 3B in a second position.

FIG. 9 is a cutaway side view of the coupler device and a duodenum scope of FIGS. 3A and 3B in a third position.

FIG. 10 is an enlarged side view of the working channel extension with membrane of the coupler device of FIGS. 3A and 3B.

FIG. 11 is a top-down view of the coupler device of FIGS. 3A and 3B.

FIG. 12 is a cutaway view of another exemplary embodiment of a coupler device of the present disclosure.

FIG. 13 is a cutaway side view of the coupler device of FIG. 12.

FIG. 14 is a cutaway side view of the coupler device of FIG. 13 in use with a duodenum scope.

FIG. 15 is an enlarged side view of an exemplary embodiment of a working channel extension of the present disclosure.

FIG. 16 is another enlarged side view of the working channel extension of FIG. 15.

FIG. 17A is a perspective view of the working channel extension of FIG. 15.

FIG. 17B shows the working channel extension of FIG. 17A in use with an instrument.

FIG. 18 is a perspective top-down view of the coupler device of FIG. 3 with a locking feature.

FIG. 19 is a perspective view of another exemplary embodiment of a working channel extension of the present disclosure.

FIGS. 20A-20C illustrate several embodiments of anti-reflective coatings on a visualization section of the coupler device according to the present disclosure.

FIG. 21 illustrates an anti-fogging coating on a visualization section of the coupler device according to the present disclosure.

### DETAILED DESCRIPTION

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

While the following disclosure is primarily directed to an optical coupler or companion device for use with an optical image endoscope, it should be understood that the features of the presently described device may be readily adapted for use with a variety of reusable or disposable endoscopic scopes, instruments and devices. The optical coupler of the present invention may also be used as part of a surgical kit that includes a variety of different instruments or devices for use in a procedure. Such a kit is described in co-pending, commonly assigned, US patent application titled Medical Device Kit with Endoscope Accessory, filed Dec. 17, 2019, the complete disclosure of which is incorporated herein by reference in its entirety for all purposes. One example of a coupler device for use with the present invention is described in co-pending, commonly assigned PCT Patent Application No. PCT/US2016/043371, filed Jul. 21, 2016, which claims the benefit of U.S. Provisional Application No. 62/195,291, filed Jul. 21, 2015, the entire disclosures of which are incorporated herein by reference for all purposes.

The term “endoscope” in the present disclosure refers generally to any scope used on or in a medical application, which includes a body (human or otherwise) and includes, for example, a laparoscope, duodenoscope, arthroscope, colonoscope, bronchoscopes, enteroscope, cystoscope, laparoscope, laryngoscope, sigmoidoscope, thoracoscope, cardioscope, and saphenous vein harvester with a scope, whether robotic or non-robotic.

When engaged in remote visualization inside the patient's body, a variety of scopes are used. The scope used depends on the degree to which the physician needs to navigate into the body, the type of surgical instruments used in the procedure and the level of invasiveness that is appropriate for the type of procedure. For example, visualization inside the gastrointestinal tract may involve the use of endoscopy in the form of flexible gastroscopes and colonoscopes, endoscopic ultrasound scopes (EUS) and specialty duodenum scopes with lengths that can run many feet and diameters that can exceed 1 centimeter. These scopes can be turned and articulated or steered by the physician as the scope is navigated through the patient. Many of these scopes include one or more working channels for passing and supporting instruments, fluid channels and washing channels for irrigating the tissue and washing the scope, insufflation channels for insufflating to improve navigation and visualization and one or more light guides for illuminating the field of view of the scope.

Smaller and less flexible or rigid scopes, or scopes with a combination of flexibility and rigidity, are also used in medical applications. For example, a smaller, narrower and much shorter scope is used when inspecting a joint and performing arthroscopic surgery, such as surgery on the shoulder or knee. When a surgeon is repairing a meniscal tear in the knee using arthroscopic surgery, a shorter, more rigid scope is usually inserted through a small incision on one side of the knee to visualize the injury, while instruments are passed through incisions on the opposite side of the knee. The instruments can irrigate the scope inside the knee to maintain visualization and to manipulate the tissue to complete the repair

Other scopes may be used for diagnosis and treatment using less invasive endoscopic procedures, including, by way of example, but not limitation, the use of scopes to inspect and treat conditions in the lung (bronchoscopes), mouth (enteroscope), urethra (cystoscope), abdomen and peritoneal cavity (laparoscope), nose and sinus (laryngoscope), anus (sigmoidoscope), chest and thoracic cavity (thoracoscope), and the heart (cardioscope). In addition, robotic medical devices rely on scopes for remote visualization of the areas the robotic device is assessing and treating.

These and other scopes may be inserted through natural orifices (such as the mouth, sinus, ear, urethra, anus and vagina) and through incisions and port-based openings in the patient's skin, cavity, skull, joint, or other medically indicated points of entry. Examples of the diagnostic use of endoscopy with visualization using these medical scopes includes investigating the symptoms of disease, such as maladies of the digestive system (for example, nausea, vomiting, abdominal pain, gastrointestinal bleeding), or confirming a diagnosis, (for example by performing a biopsy for anemia, bleeding, inflammation, and cancer) or surgical treatment of the disease (such as removal of a ruptured appendix or cautery of an endogastric bleed).

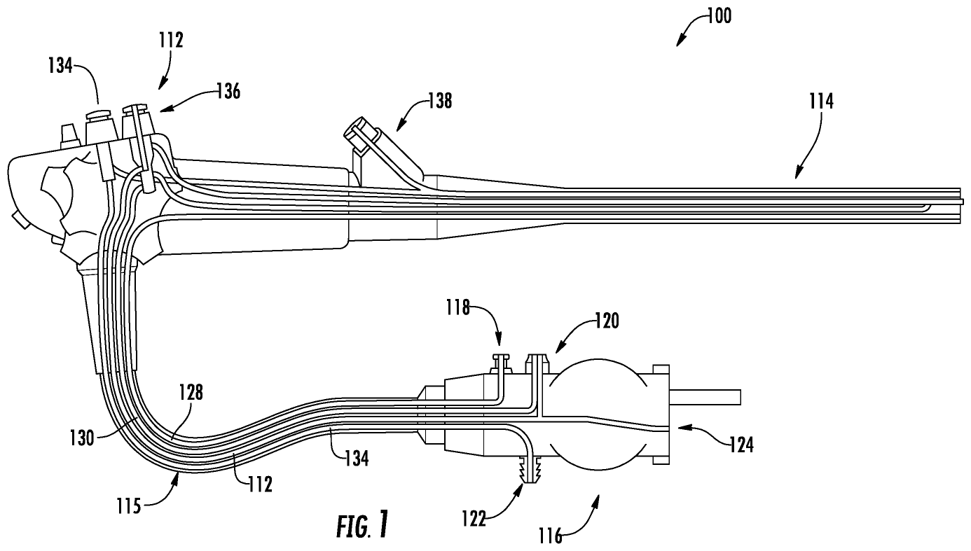

Referring now to FIG. 1, the present disclosure may include an optical viewing endoscope of the type described above. A representative endoscope 100 for use with the present disclosure includes a proximal handle 112 adapted for manipulation by the surgeon or clinician coupled to an elongate shaft 114 adapted for insertion through a natural orifice or an endoscopic or percutaneous penetration into a body cavity of a patient. Endoscope 100 further includes a fluid delivery system 116 coupled to handle 112 via a universal cord 115. Fluid delivery system 116 may include a number of different tubes coupled to internal lumens within shaft 114 for delivery of fluid(s), such as water and air, suction, and other features that may be desired by the clinician to displace fluid, blood, debris and particulate matter from the field of view. This provides a better view of the underlying tissue or matter for assessment and therapy. In the representative embodiment, fluid delivery system 116 includes a water-jet connector 118, water bottle connector 120, a suction connector 122 and an air pipe 124. Water-jet connector 118 is coupled to an internal water-jet lumen 126 that extends through handle 112 and elongate shaft 114 to the distal end of endoscope 100. Similarly, water jet connector 118, water bottle connector 120, suction connector 122 and air pipe 124 are each connected to internal lumens 128, 130, 132, 134 respectively, that pass through shaft 114 to the distal end of endoscope 100.

Endoscope 100 may further include a working channel (not shown) for passing instruments therethrough. The working channel permits passage of instruments down the shaft 114 of endoscope 100 for assessment and treatment of tissue and other matter. Such instruments may include cannula, catheters, stents and stent delivery systems, papillotomes, wires, other imaging devices including mini-scopes, baskets, snares and other devices for use with a scope in a lumen.

Proximal handle 112 may include a variety of controls for the surgeon or clinician to operate fluid delivery system 116. In the representative embodiment, handle 112 include a suction valve 135, and air/water valve 136 and a biopsy valve 138 for extracting tissue samples from the patient. Handle 112 will also include an eyepiece (not shown) coupled to an image capture device (not shown), such as a lens and a light transmitting system. The term “image capture device” as used herein also need not refer to devices that only have lenses or other light directing structure. Instead, for example, the image capture device could be any device that can capture and relay an image, including (i) relay lenses between the objective lens at the distal end of the scope and an eyepiece, (ii) fiber optics, (iii) charge coupled devices (CCD), (iv) complementary metal oxide semiconductor (CMOS) sensors. An image capture device may also be merely a chip for sensing light and generating electrical signals for communication corresponding to the sensed light or other technology for transmitting an image. The image capture device may have a viewing end—where the light is captured. Generally, the image capture device can be any device that can view objects, capture images and/or capture video.

In some embodiments, endoscope 100 includes some form of positioning assembly (e.g., hand controls) attached to a proximal end of the shaft to allow the operator to steer the scope. In other embodiments, the scope is part of a robotic element that provides for steerability and positioning of the scope relative to the desired point to investigate and focus the scope.

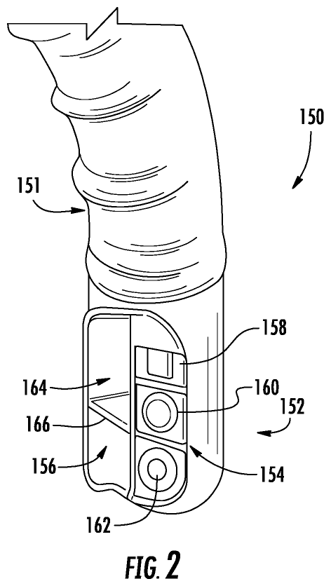

Referring now to FIG. 2, a distal end portion of a side viewing endoscope 150 (e.g., a duodenoscope or EUS) will now be described. As shown, scope 150 includes an elongate flexible shaft 151 with distal end portion 152 having a viewing region 154 and an instrument region 156, both of which face laterally or to the side of the longitudinal axis of shaft 151. Viewing region 154 includes an air nozzle port 158, a camera lens 160 and a light source 162 for providing a view of the target site in the patient. Instrument region 156 includes an opening 164 coupled to a working channel (not shown) within shaft 151 of scope 150. Opening 164 is configured to allow passage of instruments from the working channel of scope 150 to the target site. Scope 150 also preferably includes an articulation mechanism for adjusting the angle that the instruments pass through opening 164. In the exemplary embodiment, the articulation mechanism comprises an elevator 166, although it will be recognized by those skilled in the art that the articulation mechanism may include a variety of other components designed to articulate the instrument angle, such as a cable extending through shaft 151 or the like.

Of course, it will be recognized that the coupler devices of the present invention may also be used for forward viewing scopes, such as a gastroscope, colonoscope or the like. A more complete description of such a device can be found in commonly-assigned co-pending U.S. patent application titled Endoscope Accessory and Medical Device Kit, filed Dec. 17, 2019, the complete disclosure of which is incorporated herein by reference for all purposes.

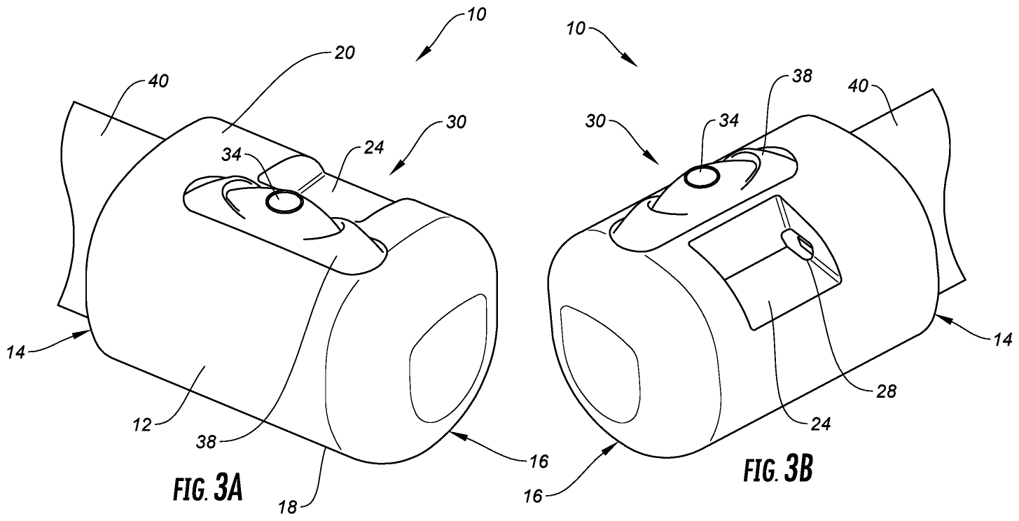

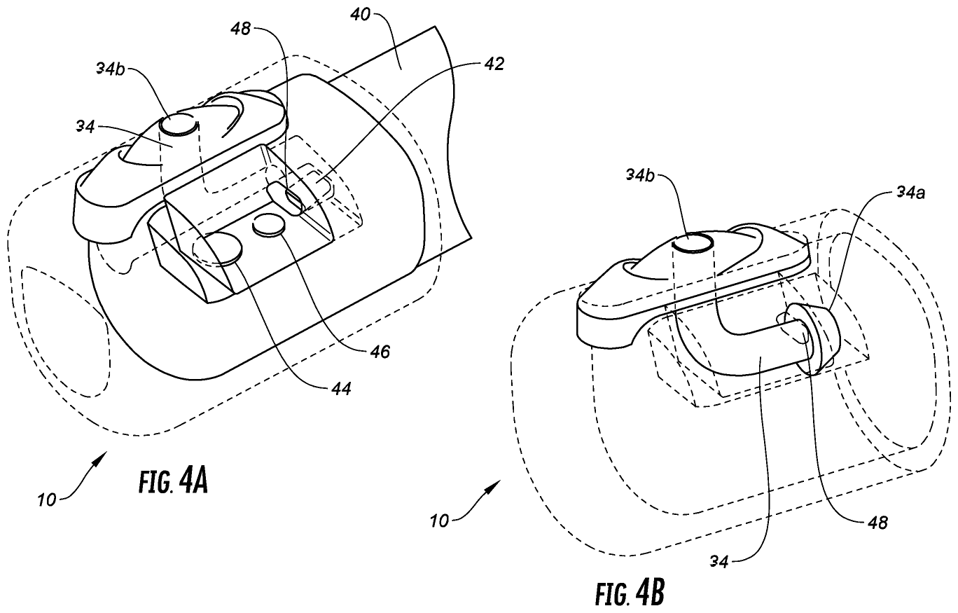

FIGS. 3A and 3B illustrate an exemplary embodiment of a coupler device 10 of the present disclosure. The coupler device 10 serves as an accessory component for currently existing endoscopes. The device seals and covers infection prone areas of the scope to prevent ingress of debris, fluid, or other unwanted matter that could lead to bacterial contamination and decreased performance of the scope.

In certain embodiments, the coupler device 10 provides a flexible working channel for instruments to be inserted into the scope. The flexible working channel can be angularly adjustable with ease. As shown, in the preferred embodiments, the coupler device 10 may be used with a duodenum scope 40 or other side-viewing scope instrument. It is understood, of course, that the coupler device 10 may be adapted for use with end viewing scopes as well. In addition, the coupler device 10 of the present disclosure can be used with all types of scopes for different medical applications. The duodenum scope 40 shown here is merely for illustrative purposes.

Of course, it will be recognized that the instruments passing through the scope may be articulated by a variety of different mechanism. For example, in some embodiments, the device may have multiple cables so the angle of exit can be articulated in multiple directions, including in different quadrants, unlike with the current endoscope elevators, which can only deflect and therefore redirect an instrument in a single axis due to the limited travel of endoscope elevators, which can only be raised or lowered, but not moved from side to side or articulated into other quadrants. In some embodiments, the cable(s) may be attached directly to the working channel extension or to other devices that can be articulated and cause the working channel extension to change its angle of exit, including, for example, a dowel underneath the working channel extension, but encased in the device that can be advanced forward and backward to move the working channel extension as the cable is advanced and retracted. In some embodiments, the articulation ability of the coupler device may be created with an elevator embedded in the coupler device, which is disposable and therefore thrown away after the procedure.

The articulation ability of the coupler device may also take place with elements that do not involve cables, including for example, piezo electric materials, micro motors, organic semiconductors, and electrically activated polymers. In some embodiments, the articulation ability of the coupler device may also take place with the transfer of force to the working channel extension or an embedded elevator through interlocking connectors that transfer force, wires that twist, slidable sheaths, and memory metals that change shape through the transfer of temperature. In some embodiments, the device includes a power connector or motors to deliver energy, including electromagnetic energy, to the device to cause a transfer in force to change the angle of exit from the coupler device as an instrument is passed through the device, or in advance of passing an instrument through the device. This transfer of force can include causing the device to rotate as it exits the working channel extension. The device may be navigated and articulated by the user directly, or as part of a robotic system in which the users input is translated through the system through various means, including cables, power connectors, motors, electromagnetic energy, slidable sheaths, haptics, computer-guided and directed input, and other means to direct and guide the device to its intended location, including to specific diagnosis and treatment objectives in a patient, or in non-medical applications, to a desired remote location.

As FIGS. 3A and 3B illustrate, the coupler device 10 may comprise a main body 12, a proximal end 14 and a distal end 16, a lower surface 18 and an upper surface 20. The proximal end 14 attaches onto a working end of a duodenum scope 40, extending the working end portion of the scope 40. The upper surface 20 may include a lens and light guide 24 and a scope washer opening 28, which is used to push fluid across the scope camera to wash debris off the camera and is also used to push air across the camera to dry the camera and insufflate the patient's gastrointestinal tract. Upper surface 20 may further include an open area over lens and light guide 24 and scope washer opening 28 to facilitate viewing the target site and to allow egress of fluid from scope washer opening 28 into the target site (and/or egress of air that may be passed over light guide 24 to dry the camera or that may be passed into the target site to insufflate a portion of the site). In addition, the upper surface 20 includes a flexible working channel region 30 that includes a flexible working channel extension 34 that is surrounded by a flexible membrane 38. This flexible membrane 138 serves as a protective hood or covering for the working end of the coupler device 10, providing for flexible articulation while sealing out debris, fluid, bacteria or other unwanted matter.

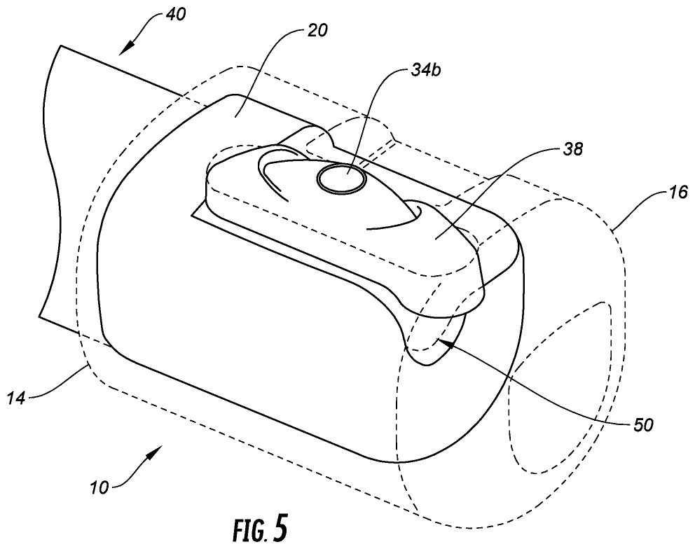

As shown in FIGS. 4A and 4B, the duodenum scope 40 may comprise a light guide 44, lens 46 and washer opening 48. The coupler device 10 cooperates with each of these components of the scope 40 to provide a fully functioning scope. The coupler device 10 does not interfere with the scope's ability to emit a clear image, but instead reduces the risk of contamination with each use. This benefit is achieved by providing a coupler device 10 which attaches to the working end components of the scope 40, and seals around the working end.

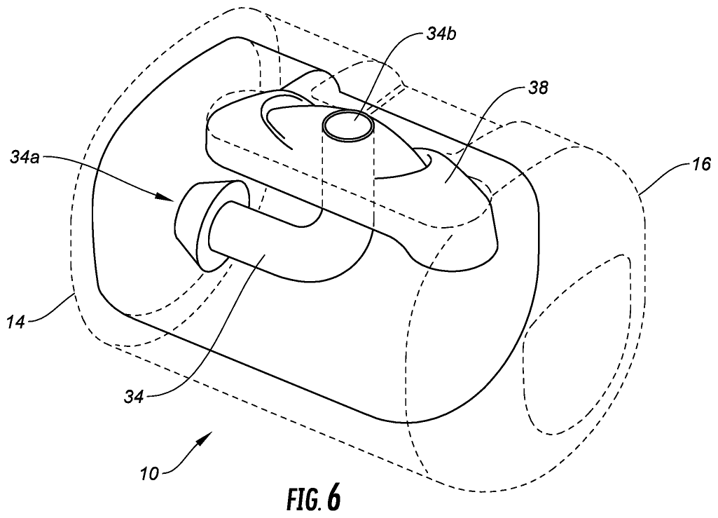

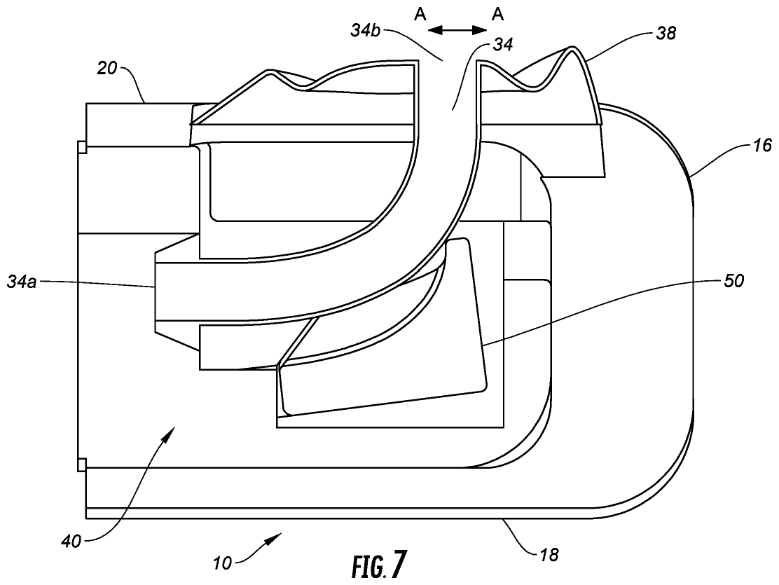

As further shown in FIGS. 3A, 3B, 4A, 4B, 5 and 6, the coupler device 10 provides an extension of the scope's working channel 42. The working channel extension 34 of the coupler device 10 in FIG. 3 is flexible and may contact the scope's working channel 42 by a sealed connection, as shown in FIG. 6, at the proximal end 34a of the working channel extension. The distal end 34b of the working channel extension 34 serves as an exit portal for instruments to pass through the scope 40 to reach different areas of the body.

Additionally, the coupler device 10 provides a further seal around the elevator 50 of the scope. Because the coupler device 10 seals the elevator 40, risk of debris influx, fluids, bacteria and other matter build up behind the elevator and working channel is reduced significantly. This influx of debris, bacteria and other matter is believed to be the reason for drug resistant infections with current scopes today. While preventing influx, the coupler device 10 advantageously maintains flexibility to move the working channel extension 34.

In use, the scope's working channel extension 34 permits passage of instruments down the scope working channel 42 and through and out the working channel extension 34 of the device 40 for assessment and treatment of tissue and other matter. Such instruments may include cannula, catheters, stents and stent delivery systems, papillotomes, wires, other imaging devices including mini-scopes, baskets, snares and other devices for use with a scope in a lumen. This working channel extension 34 is flexible enough that the elevator 50 of the scope 40 can raise and lower the working channel extension 34 so that instruments can be advanced down and out of the working channel extension distal end (or exit portal) 34b of the scope 40 at various angles, or be raised and lowered by a cable or other means to articulate the working channel extension 34.

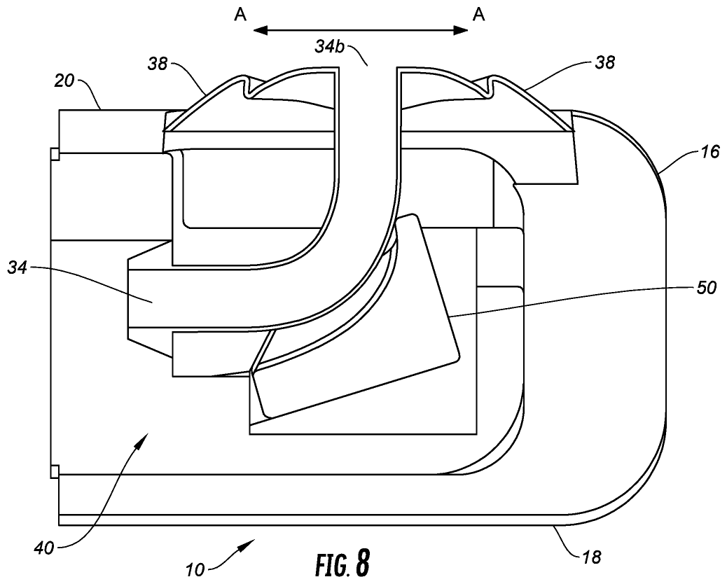

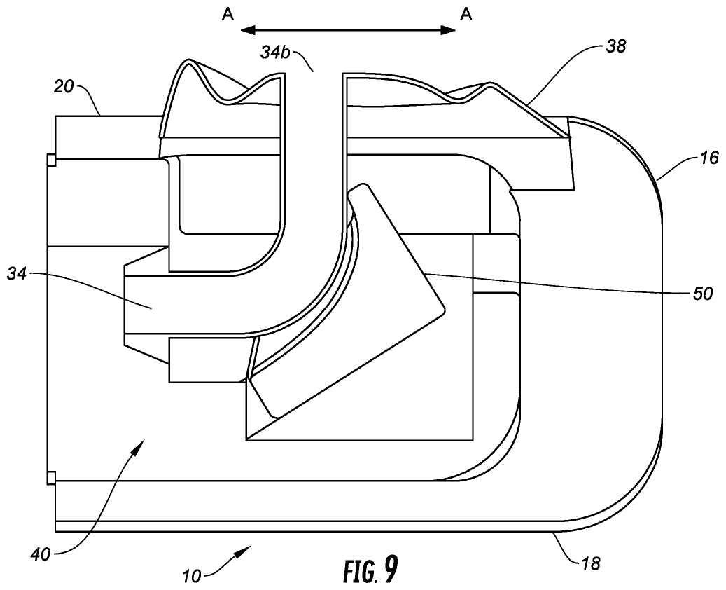

As FIGS. 7 to 9 illustrate, in use when the elevator 50 of the scope 40 is actuated, the flexible working channel extension 34 of the coupler device moves or adjusts to this actuation, along the direction A-A. In FIG. 7, the elevator 50 is raised slightly, creating a hinged ramp or shoulder that pushes the working channel extension 34 a corresponding angle and shifts the exit portal or distal end 34b of the working channel extension to the left. In FIG. 8 the elevator is raised higher than in FIG. 7, such that the distal end 34b of working channel extension 34 is likewise shifted further to the left in comparison to FIG. 7, while FIG. 9 shows the elevator 50 raised even higher and the distal end 34b of working channel extension 34 moved to the left even further in comparison to FIGS. 7 and 8.

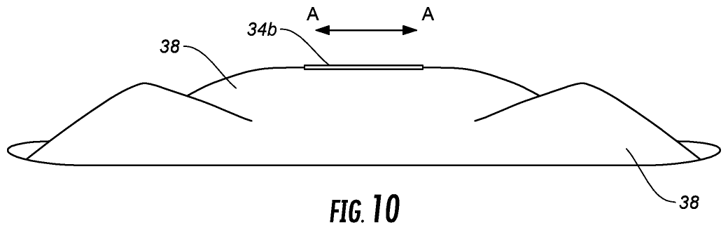

As FIG. 10 shows, the ability of the distal end 34b of working channel extension 34 to shift along the width of the working channel region 30 of the coupler device 10 is in part due to the fact that the distal end 34b is itself attached to a flexible membrane 38. This flexible membrane 38 comprises a plurality of loose folds or creases, allowing the excess material to stretch and bend as the elevator actuation forces the working channel extension to bend and shift in response. In addition, the flexible membrane 38 acts as a protective cover or hood for the working channel region 38, preventing the ingress of fluids, debris, or other unwanted matter from getting inside the scope 40 and causing a bacterial contamination or the infusion of other unwanted fluid, debris or particulate matter.

It is contemplated that the coupler device 10 of the present disclosure may be configured for single, disposable use, or it may be configured for reuse. The coupler device 10 may be made of any biocompatible material, such as for example, silicone or another elastic or polymeric material. In addition, the material may be transparent. As shown in FIG. 11, the coupler device 10 may be formed of a transparent material to provide a transparent covering of the scope camera and light source, thereby allowing unhindered performance of the scope 40.

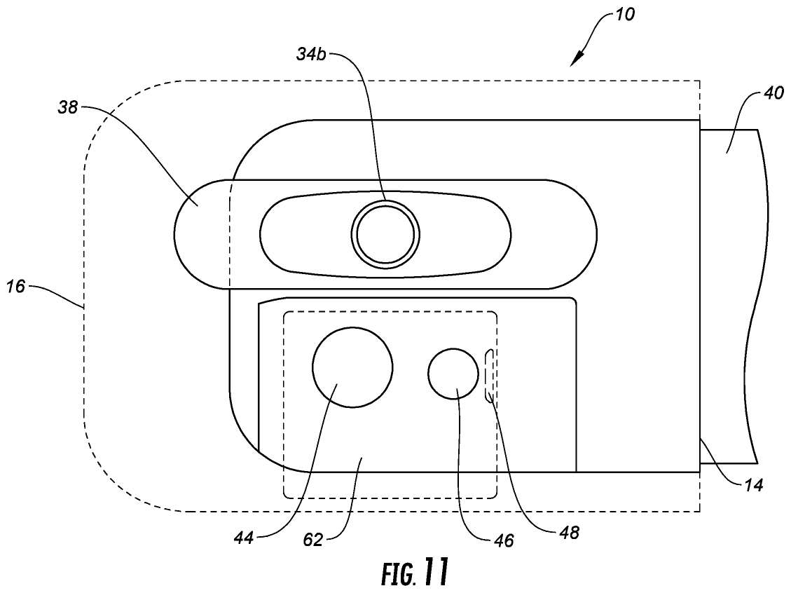

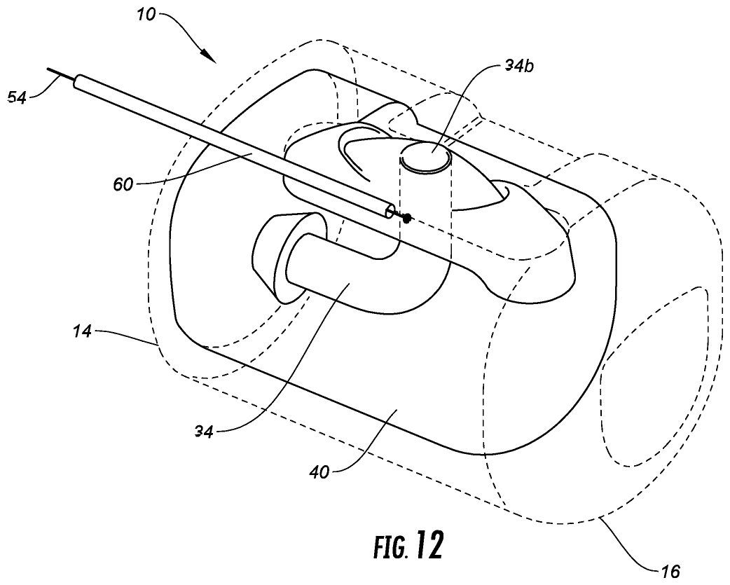

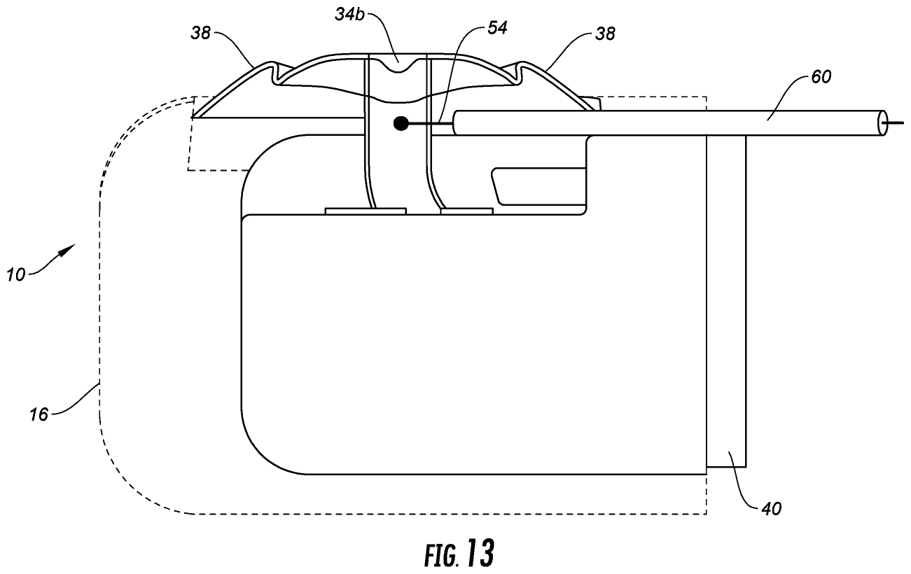

FIGS. 12 to 14 show another exemplary embodiment of a coupler device 10 of the present disclosure. In this embodiment, the coupler device 10 is adapted for use with scopes that are actuated by cable and eliminates the need for the elevator component. As illustrated, the coupler device 10 maintains the same structural features as previously described, but now includes a further disposable external sheath 60 that can receive an interior actuating cable 54 of the scope. This cable 54 can be detached from the elevator and reattached to the flexible working channel extension 34 of the coupler device 10. The elevator is no longer needed in this embodiment, as actuation of the cable effects movement of the working channel extension 34. The external sheath 60 may be configured to attach directly to the scope 40, such as by winding around the outside of the scope or by a friction fit connection. In embodiments, multiple cables may be included in one or more sheaths to provide for articulation in other quadrants than the single axis articulation with elevators in current duodenoscopes.

In other embodiments, the coupler device 10 may also include a closable port (i.e., self-sealing) that allows for the injection of anti-adhesion, anti-bacterial, anti-inflammatory or other drug or infusible matter that prevents the adherence or colonization of bacteria on the scope. An applicator may be provided that is integrated into the coupler device 10 with a port for delivery of the infusible matter. Alternatively, the applicator may be separate from the coupler device 10 and applied to the distal end of the scope 40. The infusible matter may include forms of silver, including in a gel or other solution, platinum, copper, other anti-adhesion, anti-bacterial, anti-inflammatory or other drug or infusible matter that is compatible with the scope and coupler device materials and biocompatible for patient use.

In one exemplary embodiment, the device includes an anti-infective material. In another exemplary embodiment, the device includes an anti-infective coating. In still another embodiment, the device includes a coating that is hydrophobic. In yet another embodiment, the device is superhydrophobic. In even still another embodiment, the device is anti-infective and hydrophobic. Further yet in another embodiment, the device is anti-infective and superhydrophobic. In further still another exemplary embodiment, anti-inflammatory coatings are incorporated into the device. The anti-inflammatory coatings may also be hydrophilic.

In one exemplary embodiment, the device 10 may include a silver ion coating. In another embodiment, the device 10 may have a silver hydrogel applied, infused, or made part of the device 10 in the area that covers or goes around the scope elevators. In addition to silver having antimicrobial properties, silver can also conduct electricity. Thus, in still another embodiment, the device 10 may include an electrical wire or other power transmission point to enable the creation of an electric field across the silver ion coating to improve the ability of the silver ion coating to prevent infection. In some embodiments, the electrical wire or other power transmission point may also apply to other antimicrobial and conductive materials, including platinum and copper.

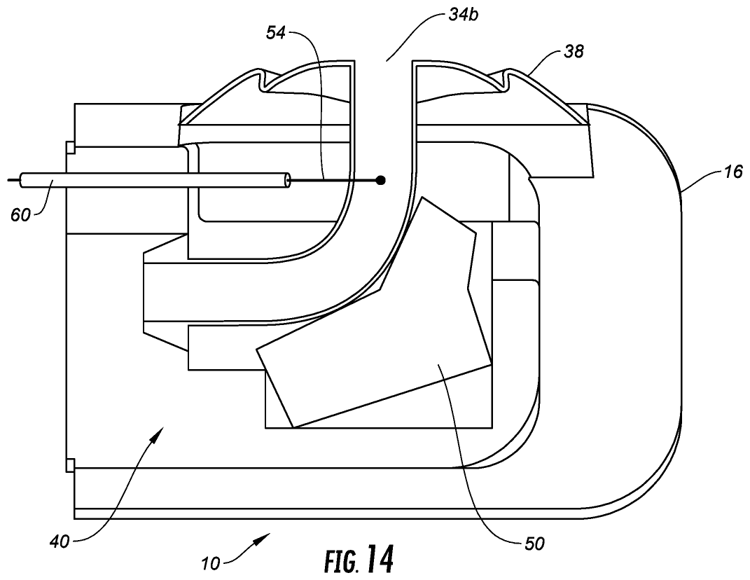

FIGS. 15 and 16 show another embodiment of the working channel extension 234 of the present disclosure. As contemplated, the working channel extensions may comprise a combination of different materials. For example, as shown in FIG. 15, the working channel extension 234 may be formed of multiple elastic materials joined to a biocompatible metal. In some embodiments, one of the elastic materials may be PTFE and another elastic material may be a biocompatible elastic material that covers the biocompatible metal. In the example of FIG. 15, the working channel extension 234 may comprise an inner elastic material 210 and an outer elastic material. The outside of the working channel extension 234 may include a biocompatible metal 230, which may take the form of a coil or winding 232. In one embodiment, the biocompatible metal may be encapsulated by one or more of the elastic materials.

In FIG. 16, the outer biocompatible elastic material 220 is formed to create a gasket 222 to seal the proximal end of the working channel extension against 234 the working channel of an endoscope, creating a seal to prevent the intrusion of unwanted bacteria, biomatter and other material into this sealed area.

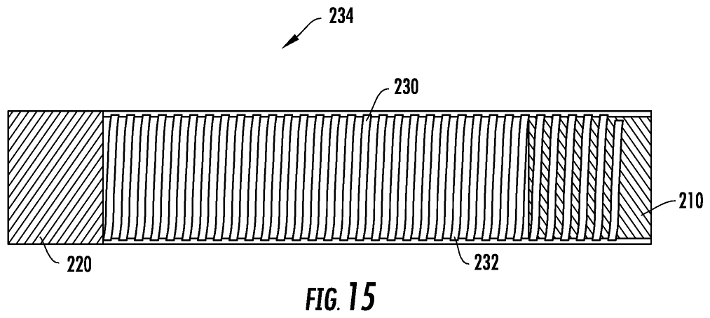

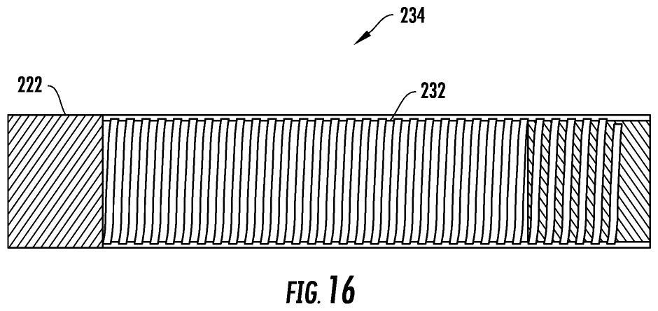

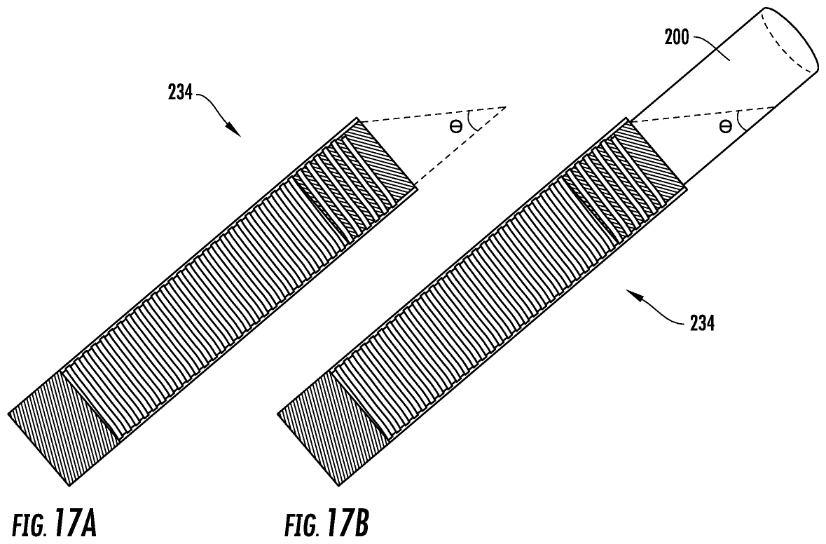

In FIG. 17A, a working channel extension 234 is shown with an adjustable angle of exit θ for locking an instrument 200 in place. In this embodiment, when the angle of exit θ is adjusted, it creates compressive force in the working channel 234, locking an instrument 200 in place, as shown in FIG. 17B. This can be used to fixate an instrument while a wire is advanced through the instrument, or to fixate a wire, while a second instrument is exchanged over the wire.

In FIG. 18, an alternative embodiment is shown for locking an instrument 200 in place. In this embodiment, the working channel extension 234 is raised to a point in which the instrument 200 in the working channel extension 234 is compressed against a lock 180 on the device 100, causing a change in the angle of exit of the working channel extension 234 and locking the instrument 200 in a fixated place in the working channel extension 234.

In FIG. 19, an alternative embodiment of the working channel extension 234 is shown with a flange 268 for attaching the working channel extension to the membrane material 38 that is part of the device 10.

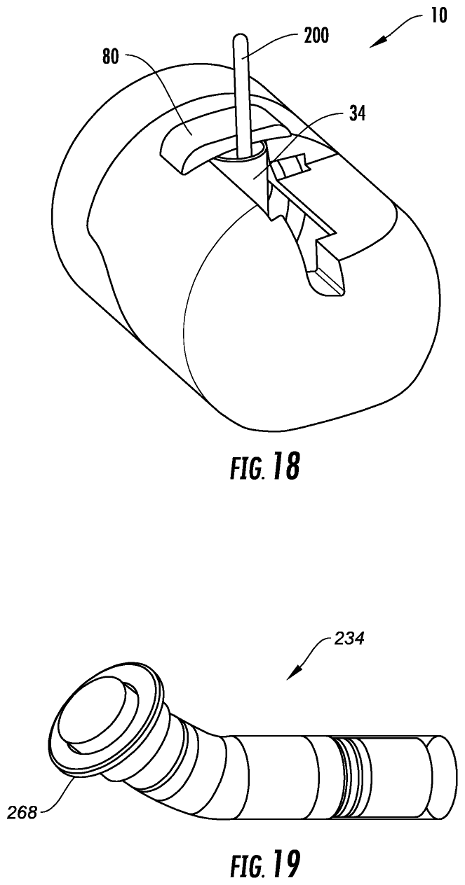

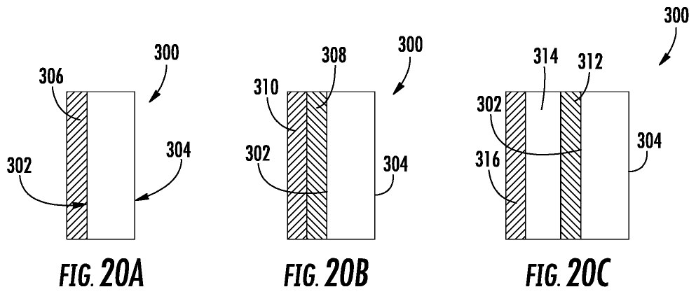

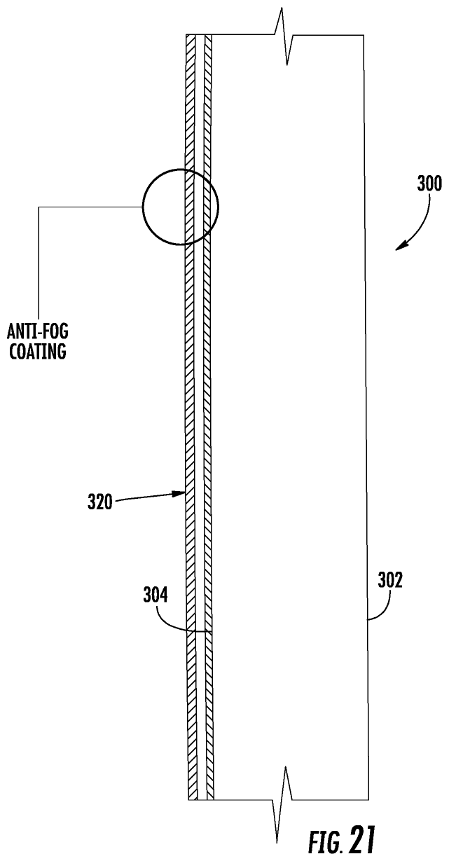

FIGS. 20A-20C illustrate several embodiments of optical layers disposed on, or within, a visualization section 300 of one of the coupler devices of the present disclosure. Alternatively, the optical layers shown in FIGS. 20A-20C may be disposed on the camera lens of an endoscope. In one embodiment, visualization section 300 preferably extends along a portion of upper surface 20 of main body 12 of coupler device 10 opposite light guide 44 and lens 46. Alternatively, visualization section 300 may be positioned directly over light guide 44 and lens 46. Visualization section 300 may comprise any suitable material, such as glass, polycarbonate, acrylic, a clear gel or silicone, or other material with sufficient optical clarity to transmit an image.

The visualization section may comprise a substantially transparent film, sheet, layer, coating, wall or other substrate positioned on the coupler device so as to allow light to pass therethrough. Alternatively, the visualization section may comprise an area within the main body of the optical coupler comprising a substantially transparent material, such as a clear gel or similar material that will allow light to pass therethrough.

In certain embodiments, the camera lens and light guide are directed laterally from the shaft of the endoscope (i.e., side viewing scopes) and the visualization section is disposed on a lateral surface of the coupler device such that light from the light guide passes therethrough to the target site. In other embodiments, the camera lens and light guide may be directly longitudinally from the shaft of the endoscope (i.e., end viewing scopes) and the visualization section is disposed on a distal surface of the coupler device. In still other embodiments, the coupler device or the endoscope may comprise mirrors or other light reflecting surfaces to redirect light passing to and from the camera and light guide so as to “align” the visualization section with the light guide and camera lens. In this latter embodiment, the visualization section may not necessarily be directly opposite the camera and light guide so long as the mirrors or other redirecting surfaces are configured to pass the light through the visualization section and to the target site.

In alternative embodiments, visualization section 300 comprises a visualization section extending within the main body of coupler that comprises a substantially clear gel material, such as silicone, silicone elastomers, epoxies, polyurethanes and mixtures thereof. Alternatively, visualization section may comprise a material selected from hydrogels, such as polyvinyl alcohol, poly(hydroxyethyl methacrylate), polyethylene glycol, poly(methacrylic acid) and mixtures thereof. The material for visualization section may also be selected from albumin based gels, mineral oil based gels, polyisoprene or polybutadiene. A more complete description of suitable clear gels suitable for the present invention is discloses in U.S. Pat. No. 8,905,921, the complete disclosure of which is incorporated herein by reference in its entirely for all purposes.

As shown in FIG. 20A, visualization section 300 comprises an inner surface 302 facing light guide 44 and lens 46 and an opposite outer surface 304 facing the target site. In one embodiment, an optical layer 306 is disposed on inner surface 302 of visualization section 300. Of course, optical layer 306 may be disposed on outer surface 304, or on both inner and outer surfaces 302, 304. Alternatively, optical layer 306 may be disposed within visualization section 300. Optical layer 306 preferably comprises an anti-reflecting material that reduces the overall reflection of light passing through optical layer 306, while substantially maintaining the transparency of light therethrough. In preferred embodiments, optical layer 306 comprises a different material than visualization section 300, particularly a material having a different index of refraction.

Optical layer 306 is preferably a thin substantially transparent film that provides a double interface to light, such that optical layer 306 and visualization section 300 generate two reflected waves. The optical layer 306 creates an interference effect with visualization section 300, thereby reducing the total percentage of reflected light without substantially reducing the transparency of the visualization section. If these reflected waves are out of phase, they partially or totally cancel each other out, thereby reducing the overall amount of light that is reflected from visualization section 300. In certain embodiments, optical layer 306 has a thickness of less than about 100 nanometers, preferably about 50 nanometers, and has an index of refraction less than visualization section 300. This causes the two reflections to be substantially out of phase with each other to maximize the amount of reflected light that is canceled out without substantially reducing the overall transparency of visualization section 300 and optical layer 306.

In certain embodiments, optical layer 306 comprises a material that is substantially non-reflective at a wavelength in the middle range of the visible spectrum (i.e., 400 to 700 nanometers). Suitable materials for optical layer 306 may include magnesium fluoride, cerium fluoride, zinc oxide, aluminum oxide and the like. In an exemplary embodiment, optical layer comprises magnesium fluoride.

Referring now to FIG. 20B, another embodiment of the present invention includes two optical layers 308, 310 disposed on inner surface 302 of visualization section 300. Each of the optical layers 308, 310 preferably comprises a different material with a different index of refraction as visualization section 300. In an exemplary embodiment, one of the optical layers 308, 310 comprises magnesium fluoride and the other optical layer comprises a suitable material, such as cerium fluoride, zinc oxide, aluminum oxide and the like.

FIG. 20C illustrates yet another embodiment of the present disclosure with three optical layers 312, 314, 316 on one of the surfaces of visualization section 300. As with the previous embodiment, each of the optical layers 312, 314, 316 comprises a different material with a different index of refraction. In an exemplary embodiment, one of the optical layers comprises magnesium fluoride, one of them comprises zinc oxide and the third optical layer comprises either aluminum oxide or cerium fluoride.

In yet another embodiment, optical couplers of the present disclosure may include one or more optical components for absorbing stray light that has been reflected from tissue surfaces and/or surfaces of the coupler. Light passing through the visualization section of the optical coupler tends to reflect from tissue surfaces in the patient back to the camera lens or the light source. This phenomenon may occur, for example, when the operator advances the endoscope from an area of smaller volume in the patient, such as the intestines, to an area of larger volume, such as an organ (e.g., the stomach). The larger volume area will cause a larger amount of reflected light passing back into the coupler device and onto the camera lens of the scope. This reflected light creates a glare that further limits the ability of the operator to view the target site.

The optical component(s) of the present disclosure may comprise a light absorbing material positioned in, or on, one or more areas of the coupler device, such as the visualization section or the main body. For example, the light absorbing material may be disposed on main body 12, lower surface 18, upper surface 20, the open area over lens and light guide 24 and scope washer opening 28, within flexible working channel region 30, such as within, or on, working channel extension 34 or flexible membrane 38 (see FIGS. 3 and 4).

The light absorbing material is positioned to absorb reflected, scattered or stray light from the patient's tissue and/or the surfaces of the optical coupler, thereby minimizing glare that may otherwise be caused by this reflected light. The light absorbing material may be a thin black strip, square, circle, rectangle or other suitable shape positioned to absorb reflected light such that the light does not interfere with the camera lens.

Alternatively, the optical component may comprise a baffle, such as a camera baffle, positioned on, or within, the coupler device. The baffle preferably includes one or more channels or vanes positioned and configured to direct stray reflected light away from the camera lens on the endoscope. Baffles can be designed to shield the light coming from sources outside of the field of view (FOV) of the camera lens. The light outside the angular view of the system must execute multiple number of reflections on its surfaces, minimizing the intensity of the light that effectively reach the camera lens. The internal surfaces may comprise light absorbing materials or colors, e.g., blackened surfaces.

The baffle of the present disclosure may comprise a tube with vanes in internal walls. These vanes are used to reduce the intensity of light that is reflected on the walls. The baffle may, for example, be positioned on or within main body 12, or one lower surface 18 or upper surface 20. Alternatively, the baffle may be positioned within the open area over lens and light guide 24 and scope washer opening 28 or within flexible working channel region 30, such as within, or on, working channel extension 34 or flexible membrane 38.

FIG. 21 illustrates another embodiment of the present invention that includes an optical layer 320 on, or within, one of the surfaces of visualization section 300. In the preferred embodiment, optical layer 320 comprises an anti-fogging agent, treatment or material that is disposed on inner surface 304 of visualization section 300 (or within visualization section 300) to inhibit or prevent the condensation of fluid, such as water, in the form of small droplets on visualization section 300, thereby improving the view of the target site within the patient. Alternatively, optical layer 320 may be formed on the outer surface of visualization section 300, or it may be disposed directly on the camera lens of the endoscope. Optical layer 320 is preferably designed to minimize surface tension, resulting in a non-scattering film of fluid, such as water, instead of single droplets that would resemble fog and disrupt the view through visualization section 300.

Optical layer 320 may be applied to visualization section 300 as a spray solution, cream, gel or the like, or it may comprise a thin film bonded to visualization section 300, or chemicals or additives that are manufactured within visualization section 300. In one embodiment, optical layer 320 comprises a surfactant configured to minimize the surface tension of the water. Suitable surfactants for use with the present invention may comprise nonionic, anionic, cationic or amphoteric polarities and may include detergents, fatty acid soaps, dispersants or other suitable materials that reduces the force of surface tension from water molecules on the visualization section to thereby reduce the number of water droplets that form on the outer surface.

In another embodiment, optical layer 320 comprises a hydrophilic coating configured to maximize the surface energy of visualization section 300. Suitable coatings for use with the present invention include demisters, defoggers, defrosters, polymers and hydrogels, such as gelatin, colloids and nanoparticles, such as titanium dioxide, or other suitable hydrophilic materials.

In yet another embodiment, optical layer 320 comprises chemicals or additives that are added to visualization section 300 such that they exude from the inside of visualization section 300 to outer surface 304. The chemicals produce a surface that inhibits the formation of water droplets.

Hereby, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

What is claimed is:

1. A coupler device for an endoscope having a light and a camera lens, the coupler device comprising:
a main body comprising an at least partially closed distal end and a proximal end configured for attachment to a distal end portion of the endoscope;
a substantially transparent visualization section disposed between the light and the camera lens of the endoscope and a target site within the patient to allow viewing of the target site; and
an optical component on, or within, the visualization section or the main body,
the optical component being configured to reduce an amount of reflected light from a surface of the visualization section.

2. The coupler device of claim 1, wherein the optical component comprises an anti-reflective coating disposed on an inner surface of the visualization section.

3. The coupler device of claim 1, wherein the optical component comprises an optical layer configured to reduce a substantial portion of the reflected light in a visible range of light waves.

4. The coupler device of claim 1, wherein the optical component comprises a magnesium fluoride coating.

5. The coupler device of claim 1, wherein the optical component comprises two or more anti-reflecting coatings.

6. (canceled)

7. The coupler device of claim 1, wherein the visualization section comprises a substantially transparent viewing surface between the camera lens and the light and the target sight.

8. (canceled)

9. The coupler device of claim 1, wherein the visualization section comprises a substantially transparent material disposed within the main body.

10. (canceled)

11. The coupler device of claim 1 further comprising a second optical component on, or within, the visualization section, wherein the second optical component is configured to inhibit condensation of fluids on the visualization section.

12. (canceled)

13. The coupler device of claim 1 further comprising a mechanism for articulating an instrument passing through a working channel on the endoscope.

14. (canceled)

15. (canceled)

16. (canceled)

17. The coupler device of claim 1, wherein the optical component comprises a light absorbing material positioned to reduce an amount of reflected light from a surface of the coupler device.

18. The coupler device of claim 1, wherein the optical component comprises a light absorbing strip on, or within, the visualization section of the coupler device, the light absorbing strip being positioned to absorb stray light from an interior of the patient.

19. The coupler device of claim 1, wherein the optical component comprises a baffle on the coupler device, the baffle having one or more vanes configured to direct stray light away from the camera lens.

20. A coupler device for an endoscope having a light and a camera lens, the coupler device comprising:
a main body comprising an at least partially closed distal end and a proximal end configured for attachment to a distal end portion of the endoscope;
a substantially transparent visualization section disposed between the light and the camera lens of the endoscope and a target site to allow viewing of the target site; and
an optical layer on, or within, the visualization section and configured to inhibit condensation of fluid on the visualization section.

21. The coupler device of claim 20, wherein the optical layer comprises one or more chemicals applied to an outer surface of, or within, the visualization section, the chemicals being configured to inhibit condensation of water on the visualization section.

22. The coupler device of claim 20, wherein the optical layer comprises an anti-fogging film applied to a surface of the visualization section.

23. The coupler device of claim 20, wherein the optical layer comprises a surfactant configured to minimize surface tension of water on the visualization section.

24. The coupler device of claim 20, wherein the optical layer comprises a hydrophilic coating on a surface of the visualization section.

25. The coupler device of claim 20, wherein the visualization section comprises a substantially transparent viewing surface between the camera lens and the light and the target sight.

26. (canceled)

27. The coupler device of claim 20, wherein the visualization section comprises a substantially transparent material disposed within the main body.

28. (canceled)

29. The coupler device of claim 20 further comprising a second optical layer on, or within, the visualization section, the second optical layer being configured to reduce an amount of reflected light on the visualization section.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. A system for treating a patient, the system comprising:
an endoscope having a light and a camera lens;
a coupler device comprising a main body comprising an at least partially closed distal end and a proximal end configured for attachment to a distal end portion of the endoscope and a substantially transparent visualization section disposed between the light and the camera lens of the endoscope and a target site to allow viewing of the target site; and
an optical component on a surface of the camera lens configured to reduce an amount of reflected light on the camera lens.

48. The system of claim 47 further comprising a second optical component the surface of the camera lens and configured to inhibit condensation of fluid on the surface.
